(19) United States
(12) Patent Application Publication
(10) Pub. No.: US 2024/0342379 A1
(43) Pub. Date: Oct. 17, 2024

(54) SPARK GENERATOR FOR GENERATING PRESSURE, FORMULA INJECTOR COMPRISING SPARK GENERATOR, METHOD FOR GENERATING PRESSURE BY SPARK, AND METHOD FOR INJECTING FORMULA

(71) Applicant: L'Oreal, Paris (FR)

(72) Inventors: Chin Kai Lee, Kawasaki-shi (JP); Woo Ram Park, Kawasaki-shi (JP)

(73) Assignee: L'Oreal, Paris (FR)

(21) Appl. No.: 18/292,133

(22) PCT Filed: Jul. 22, 2022
(86) PCT No.: PCT/JP2022/029218

§ 371 Date: Jan. 25, 2024

(30) Foreign Application Priority Data
Jul. 27, 2021 (JP) 2021-122470
Sep. 16, 2021 (FR) 2109736

(51) Int. Cl. A61M 5/20; A61M 5/30; F02P 3/01

(52) U.S. Cl. CPC A61M 5/2053; A61M 5/30; F02P 3/01

(57) ABSTRACT

The present application provides a spark generator for generating pressure. The spark generator comprises: a main device; and a pressure chamber disposed adjacent to the main device, wherein the main device comprises: a circuit for generating a predetermined spark voltage; and an electromagnet, wherein the pressure chamber comprises: a non-compressive fluid contained therein; a fixed electrode disposed in the non-compressive fluid; a movable electrode disposed in the non-compressive fluid and separated from the fixed electrode at a predetermined distance; and a permanent magnet disposed on the movable electrode, wherein the circuit is configured to apply the spark voltage between the movable electrode and the fixed electrode, wherein the electromagnet is configured to apply a magnetic force to the permanent magnet to move the movable electrode toward the fixed electrode for generating spark between the movable electrode and the fixed electrode, wherein the pressure chamber comprises a pressure transferring structure composing a part of a wall of the pressure chamber and configured to transfer a pressure of the non-compressive fluid generated by the spark to outside of the pressure chamber.

### TECHNICAL FIELD

The present invention relates to a spark generator for generating pressure, a formula injector comprising the spark generator, a method for generating pressure by a spark, and a method for injecting a formula.

### BACKGROUND ART

For delivering a formula such as a cosmetic product and a drug into the skin, there is a demand in the medical and cosmetic fields for formula injection technology without using a needle. Delivering a formula into the skin without using a needle can maximize the effect of the formula. Minimizing the damage to the skin will result in improved safety and reduced pain. Furthermore, general consumers, who are not medical workers, can easily deliver the formula into the skin.

Many needleless formula injectors utilize a micro-jet in order to deliver a formula into the skin with minimized damage. For example, United States Patent Application, Publication No. US 2015/0265770 and U.S. Pat. No. 8,905,966 disclose formula injection devices generating a micro-jet by using laser energy.

FIG. 5 shows an example of a conventional formula injection device using laser energy. A formula injection device 100 shown in FIG. 5 comprises a pressure chamber 102 and a formula chamber 104 disposed adjacent to the pressure chamber 102.

The pressure chamber 102 is filled with a fluid 106. The fluid 106 is non-compressive and chemically inert fluid such as water. The pressure chamber 102 comprises an elastic membrane 108 constituting a part of a wall of the pressure chamber 102. The pressure chamber 102 also comprises a window 110 for transmitting laser light.

The formula chamber 104 is filled with a formula 112 to be delivered to the skin. The formula 112 is in contact with the elastic membrane 108 and separated from the fluid 106 by the elastic membrane 108. The formula chamber 104 comprises a nozzle 116 for injecting the formula 112.

Laser light 118 is injected into the pressure chamber 102 of the formula injector 100 via the window 110. Since energy of the laser light 118 is absorbed by the fluid 106, the fluid rapidly expands, and cavitation is caused under some conditions. The expansion and the cavitation generate pressure in the fluid 106. The generated pressure displaces the elastic membrane 108 toward the formula chamber 104 and is transferred to the formula 112 in the formula chamber 104. Therefore, the formula 112 is injected via the nozzle 116 as a micro-jet.

Since the formula injector 100 comprising the above configuration utilizes the laser light, a laser generator and an optical fiber for transmitting laser are needed. Therefore, miniaturization of the device is difficult. The device also consumes a large amount of power. Furthermore, all of the laser energy may not be absorbed by the fluid and the device may not be energy efficient, thus possible increasing power consumption. Since powerful lasers such as a YAG laser, are used for obtaining a desirable pressure, the operation of the device may not be easy or safe for consumers.

Accordingly, a formula injector having small dimensions and low power consumption and a device for generating pressure available for such a formula injector, which consumers can easily operate, are desired.

### SUMMARY OF INVENTION

The first embodiment of the present invention provides a spark generator for generating pressure, comprising:
a main device; and
a pressure chamber disposed adjacent to the main device,
wherein the main device comprises:
   a circuit for generating a predetermined spark voltage; and
   an electromagnet,
wherein the pressure chamber comprises:
   a non-compressive fluid contained therein;
   a fixed electrode disposed in the non-compressive fluid;
   a movable electrode disposed in the non-compressive fluid and separated from the fixed electrode by a predetermined distance; and
   a permanent magnet disposed on the movable electrode,
wherein the circuit is configured to apply the spark voltage between the movable electrode and the fixed electrode,
wherein the electromagnet is configured to apply a magnetic force to the permanent magnet to move the movable electrode toward the fixed electrode for generating a spark between the movable electrode and the fixed electrode,
wherein the pressure chamber comprises a pressure transferring structure constituting a part of a wall of the pressure chamber and configured to transfer a pressure in the non-compressive fluid generated by the spark to the outside of the pressure chamber.

In the first embodiment of the present invention, the pressure transferring structure may be an elastic membrane.

In the first embodiment of the present invention, the pressure transferring structure may be a piston.

In the first embodiment of the present invention, the pressure chamber may further comprise a selective gas filter configured to release gas produced by decomposition of the non-compressive fluid by the spark to the outside of the pressure chamber.

In the first embodiment of the present invention, the circuit may comprise a relay, and the relay may be configured to apply a current to the electromagnet and to apply the spark voltage between the movable electrode and the fixed electrode in a synchronized manner.

The second embodiment of the present invention provides a formula injector comprising:
the spark generator as described above; and
a formula chamber configured to receive pressure transferred by the pressure transferring structure,
wherein the formula chamber comprises:
   a formula contained therein; and
   a nozzle configured to inject the formula,
wherein the formula is configured to be injected from the nozzle by the pressure transferred by the pressure transferring structure.

In the second embodiment of the present invention, the pressure transferring structure may be in direct contact with the formula.

The third embodiment of the present invention provides a method for generating pressure by a spark, comprising the steps of:
applying a predetermined spark voltage between a fixed electrode disposed in a pressure chamber containing a non-compressive fluid and a movable electrode disposed in the pressure chamber and separated from the fixed electrode by a predetermined distance, and comprising a permanent magnet disposed thereon;
applying a current to an electromagnet disposed in a main device disposed adjacent to the pressure chamber and configured to apply a magnetic force to the permanent magnet to move the movable electrode toward the fixed electrode by the permanent magnet for generating a spark between the movable electrode and the fixed electrode; and
transferring pressure generated in the non-compressive fluid by the spark to the outside of the pressure chamber via a pressure transferring structure.

In the third embodiment of the present invention, the pressure transferring structure may be an elastic membrane, and the pressure may be transferred to the outside of the pressure chamber via a deformation of the elastic membrane.

In the third embodiment of the present invention, the pressure transferring structure may be a piston, and the pressure may be transferred to the outside of the pressure chamber via a movement of the piston.

In the third embodiment of the present invention, the method may further comprise releasing a gas produced by decomposition of the non-compressive fluid by the spark to the outside of the pressure chamber via a selective gas filter disposed on the pressure chamber.

In the third embodiment of the present invention, the step of applying the predetermined spark voltage between the fixed electrode and the movable electrode and the step of applying the current to the electromagnet to move the movable electrode toward the fixed electrode via the permanent magnet for generating the spark between the movable electrode and the fixed electrode may be carried out in a synchronized manner.

The fourth embodiment of the present invention provides a method for injecting a formula, comprising the steps of:
transferring pressure to a formula chamber configured to receive the pressure transferred to the outside of the pressure chamber by a method as described above via a pressure transferring structure; and
injecting a formula disposed in the formula chamber to the outside of the formula chamber via a nozzle disposed on the formula chamber by the transferred pressure.

In the fourth embodiment of the present invention, the pressure transferring structure may be in contact with the formula, and the pressure may be directly transferred to the formula.

### Advantageous Effects of Invention

According to the present invention, a formula injector having small dimensions and a low power consumption and a device for generating pressure for the formula injector, which consumers can easily operate, are embodied.

### BRIEF DESCRIPTIONS OF DRAWINGS

FIG. 1 shows a schematic diagram of a spark generator for generating pressure according to some embodiments of the present invention.

FIG. 2 shows a schematic diagram of a formula injector according to some embodiments of the present invention.

FIG. 3 shows a graph showing a relationship between a voltage and a diameter of a cavity generated by a spark.

FIG. 4 shows images of cavitation caused by the spark obtained by a high speed camera.

FIG. 5 shows a schematic diagram of a conventional formula injector.

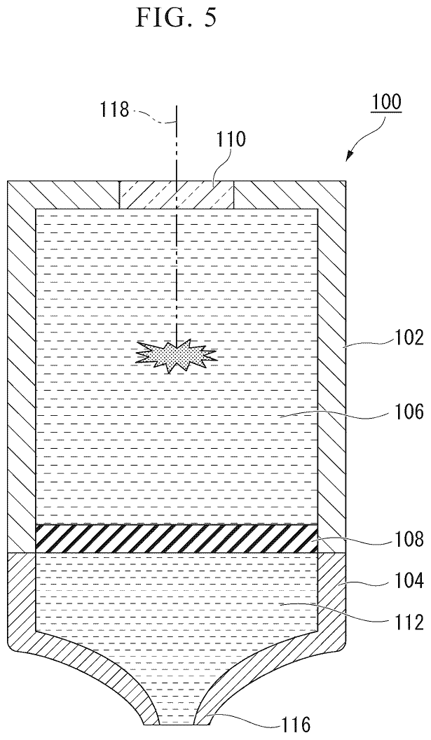

### DESCRIPTION OF EMBODIMENTS

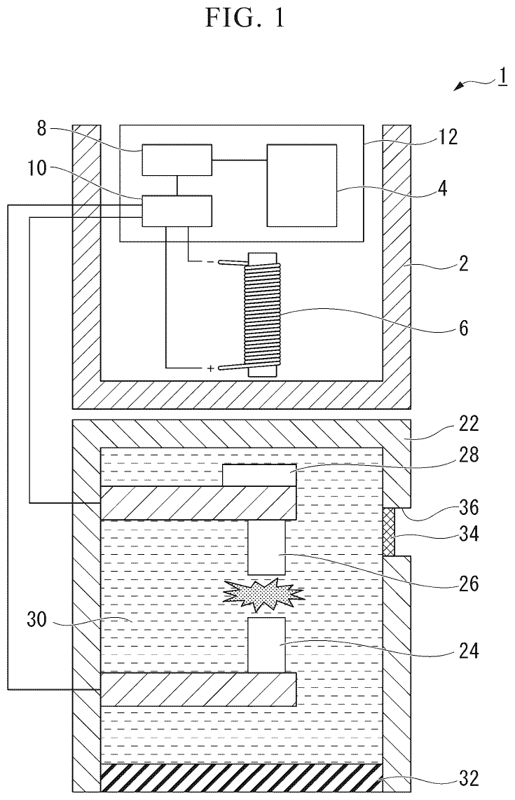

FIG. 1 shows a schematic diagram of a spark generator 1 for generating pressure according to some embodiments of the present invention. The spark generator 1 comprises a main device 2 and a pressure chamber 22 disposed adjacent to the main device 2.

The main device comprises a circuit 4 for generating a predetermined spark voltage, and an electromagnet 6. The main device 2 may optionally comprise: a capacitor 8 to which the spark voltage generated by the circuit 4 is applied for storing a charge; and a relay 10 for generating a spark by applying the spark voltage to electrodes described below. The main device 2 may comprise a power supply circuit which is not shown. The power supply circuit may be powered by a commercial power supply, or may comprise a battery not shown, for example, a lithium ion rechargeable battery installed in the spark generator 1.

The electromagnet 6 may receive a current supplied from the circuit 4 or a power circuit not shown. The supply of power to the electromagnet 6 may be controlled by the relay 10. The electromagnet 6 is disposed such that a magnetic force is applied to the pressure chamber 22 disposed adjacent to the main device 2.

At least one of the circuit 4, the electromagnet 6, the capacitor 8, the relay 10, and the power circuit not shown may be controlled by a microcontroller unit not shown. At least one of these components may be installed on a main circuit board 12.

The pressure chamber 22 comprises a fixed electrode 24 and a movable electrode 26 separated from the fixed electrode 24 by a predetermined distance therein. The movable electrode 26 may ensure its movability by, for example, a hinge mechanism or a link mechanism in order to move toward the fixed electrode 24. However, considering fabrication, a simple structure, and a restoring force moving back to the original position after moving toward the fixed electrode 24, the movable electrode 26 may preferably comprise: an elastic member such as a metal cantilever or a cantilever having a dielectric material such as a rubber, which may has a shape of a flat or curved plate; and an electrode piece. The fixed electrode 24 may also preferably comprise: an elastic member; and an electrode piece similarly to the movable electrode 26. The cantilevers of the fixed electrode 24 and the movable electrode 26 may have, for example, a shape of a flat or curved plate. The electrode pieces of the fixed electrode 24 and the movable electrode 26 may be preferably a rod or a pillar. Preferably, the electrode pieces of the fixed electrode 24 and the movable electrode 26 comprise metal rods or pillars having spherical cross sections and disposed such that these protrusions face each other. In this case, when the movable electrode 26 moves toward the fixed electrode 24, an electric field is concentrated between the protrusions, and therefore the spark may be generated with a lower voltage.

A permanent magnet 28 is disposed on the movable electrode 26 opposite the side facing the fixed electrode 24 such that the permanent magnet 28 faces the electromagnet 6 disposed in the main device 2. The fixed electrode 24 and the movable electrode 26 are electrically connected to the circuit 4 such that the spark voltage from the circuit 4 is applied. The relay 10 may be interposed between the circuit 4 and the fixed and movable electrodes 24, 26, and may control the application of the spark voltage between the fixed electrode 24 and the movable electrode 26.

As described above, the relay 10 may also control the application of the current to the electromagnet 6. Therefore, when the relay 10 is turned on, the application of the current to the electromagnet 6 and the application of the spark voltage between the fixed electrode 24 and the movable electrode 26 may be carried out in a synchronized manner.

The permanent magnet 28 faces the electromagnet 6 and is configured to repel in response to a magnetic force produced by the electromagnet 6. Therefore, when the magnetic force is applied by the electromagnet 6, the permanent magnet 28 is repelled from the electromagnet 6 and moves the movable electrode 26 toward the fixed electrode 24. When the predetermined spark voltage is applied between the movable electrode 26 and the fixed electrode 24 and when the fixed electrode 24 and the movable electrode 26 contact each other, the spark is generated between the fixed electrode 24 and the movable electrode 26. The spark voltage may be, for example, applied to the movable electrode 26, and the fixed electrode 24 may be maintained at a ground potential.

Specifically, the pressure transferring structure 32 is configured to be in contact with the formula 44 contained in the formula chamber 42, and therefore the pressure transferring structure 32 is disposed to separate the non-compressive fluid 30 from the formula 44. When the pressure is applied to the formula 44 from the pressure transferring structure 32, the formula 44 is injected to the outside of the formula chamber 42 via the nozzle 46 as a micro-jet.

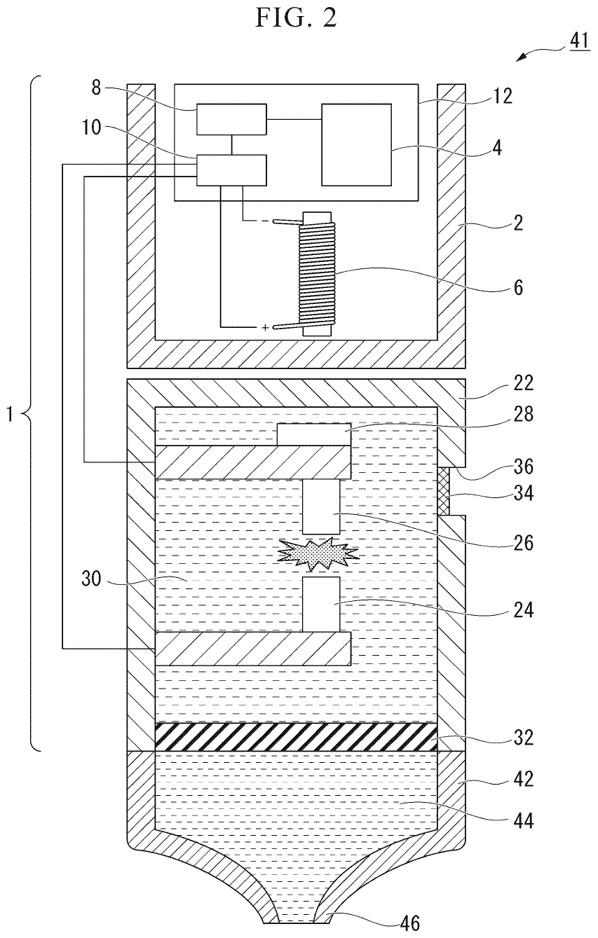

With further reference to FIGS. 1 and 2, a method for generating pressure by a spark by using the spark generator 1 configured as such, and a method for injecting a formula by using pressure generated by the spark generator 1 will be described.

The method for generating pressure carries out the step of the circuit 4 generating a spark voltage to be applied between the fixed electrode 24 and the movable electrode 26 by using, for example, a voltage booster circuit. For example, the spark voltage is preferably several hundred volts or less. More preferably, the spark voltage may be between 10 and 100 V. Even more preferably, the spark voltage may be 40 V or larger, for example, between 50 and 100 V, or between 50 and 70 V. The lower the spark voltage is, advantageously the lower the power consumption, the higher the safety, and the smaller the device can be. The spark voltage can be applied to, for example, the capacitor 8, and charges are stored.

The next step is to activate the relay 10 to apply the spark voltage 10 of the capacitor 8 between the fixed electrode 24 and the movable electrode 26 which are disposed in the pressure chamber 22 and immersed in the non-compressive fluid 30. For example, the fixed electrode 24 may be maintained at an earth potential, and the spark voltage may be applied to the movable electrode 26. An initial separation between the fixed electrode 24 and the movable electrode 26 is selected such that a spark is not generated even if the spark voltage is applied.

Next, a current is applied to the electromagnet 6 to produce a magnetic field. The magnetic field is configured to exert a repulsive force on the permanent magnet 28 disposed on the movable electrode 26 in a direction away from the electromagnet 6. When the permanent magnet 28 is pushed by the magnetic field applied by the electromagnet 6, the movable electrode 26 is displaced toward the fixed electrode 24. When the fixed electrode 24 and the movable electrode 26 contact each other, the charge stored in the capacitor 8 generates a spark between the fixed electrode 24 and the movable electrode 26. The charge emitted by the spark may be, for example, between 40 and 250 mAh, and more preferably, 100 mAh. The energy emitted by the spark may be, for example, between 1.0 and 25 J.

After the spark is finished, a step of deactivating the supply of current to the electromagnet 6 is carried out, and the emission of the magnetic force ends. Therefore, the movable electrode 26 is returned to its original position by the elastic force of the movable electrode 26. The circuit 4 may apply the spark voltage to the capacitor 8 and the charge may be stored for the next spark.

The relay 10 may apply the spark voltage between the fixed electrode 24 and the movable electrode 26 and supply the current to the electromagnet 6 synchronously or simultaneously. In this case, since only one relay 10 can carry out the application of the spark voltage and the supply of current to the electromagnet 6, the structure and the control of the device may be simplified.

The energy emitted by the spark is transferred to the non-compressive fluid 30 surrounding the fixed electrode 24 and the movable electrode 26. The non-compressive fluid 30 is locally heated and pressurized, which expands its volume or causes cavitation. Different from the conventional art using a laser, since the energy of the spark is spatially limited between the fixed electrode 24 and the movable electrode 26, a desired pressure can be caused by lower energy. The expanded voltage or the produced cavitation generates the pressure in the non-compressive fluid 30. The generated pressure propagates to the pressure transferring structure 32. Since the pressure transferring structure 32 may preferably be an elastic membrane such as rubber or silicone, or a piston, the pressure transferring structure 32 is deformed by receiving the pressure and displaces toward the outside of the pressure chamber 22. Therefore, if there is an object outside the pressure chamber 22 and in contact with the pressure transferring structure 32, the pressure is transferred to the object.

A portion of the non-compressive fluid 30 may be decomposed by the spark and may generate gas. For example, when the non-compressive fluid 30 is water, the water may be decomposed to oxygen and hydrogen. The gas is compressive and its volume is reduced by the pressure. Therefore, a part of the pressure generated in the non-compressive fluid 30 by the spark is used for the compression of the gas without being transferred to the pressure transferring structure 32. Therefore, if the gas produced by the decomposition of the non-compressive fluid 30 is left in the pressure chamber 22, that results in the pressure loss. Therefore, after the spark occurs, a step of emitting the gas from the pressure chamber 22 via an optional gas outlet 36 disposed on the pressure chamber 22 may be carried out. A selective gas filter 34 may be disposed at the gas outlet 36. The gas produced by the decomposition of the non-compressive fluid 30 passes through the selective gas filter 34 to be emitted from the pressure chamber 22, while the non-compressive fluid 30 cannot pass through the selective gas filter 34 to be maintained in the pressure chamber 22.

The formula injector 41 shown in FIG. 2 comprises a formula chamber 42 disposed adjacent to the pressure chamber 22. A formula 44 is contained in the formula chamber 42 and is in contact with the pressure transferring structure 32. Therefore, the pressure generated by the spark in the pressure chamber 22 is directly transferred to the formula 44 via the pressure transferring structure 32. Therefore, the formula 44 passes through the nozzle 46 to form a micro-jet and be emitted.

The depth of the formula 44 injected in a subject, for example, human skin depends on the pressure generated by the spark. Therefore, the depth can be set by adjusting the spark voltage. For example, the spark voltage can be adjusted between 10 and 100 V or higher. When the formula is injected into the deep portion of the skin, the spark voltage may be set higher than 100 V.

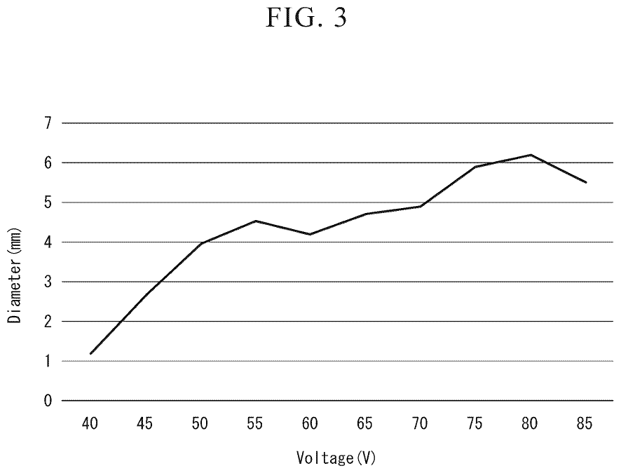

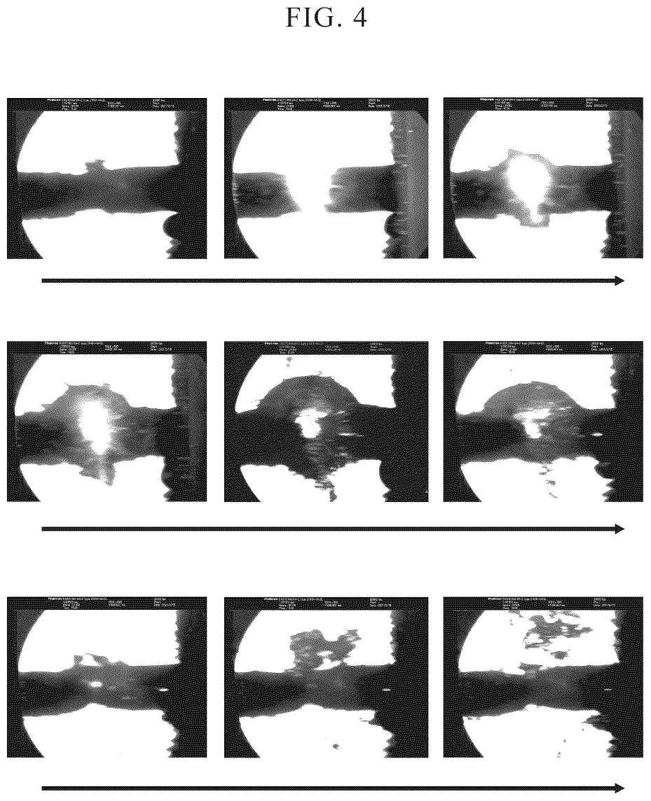

FIG. 3 shows results of measured cavitation diameters by applying various voltages between electrodes. The electrodes were immersed in the water and had a diameter of 2 mm. The separation between the electrodes was 0.2 mm. FIG. 4 shows images of cavitation captured by a high-speed camera when 65 V was applied. These results show that cavitation having a uniformed volume can be caused by applying the spark voltage between 50 and 70 V. The volume of the cavitation caused by the spark is suitable for the injection of the cosmetic product by the micro-jet.

Although specific embodiments of the present invention were described, those skilled in the art would easily understand that various changes, modifications and improvements are possible without departing from the technical spirit and scope of the present invention.

### REFERENCE SIGNS LIST

1. Spark generator
2. Main device
4. Circuit
6. Electromagnet
8. Capacitor
10. Relay
12. Main circuit board
22. Pressure chamber
24. Fixed electrode
26. Movable electrode
28. Permanent magnet
30. Non-compressive fluid.
32. Pressure transferring structure
34. Selective Gas Filter
41. Formula injector
42. Formula chamber
44. Formula
46. Nozzle
100. Conventional formula injector
102. Pressure chamber
104. Formula chamber
106. Fluid
108. Elastic membrane
110. Window
112. Formula
116. Nozzle
118. Laser

1. A spark generator for generating pressure, comprising:
a main device; and
a pressure chamber disposed adjacent to the main device,
wherein the main device comprises:
a circuit for generating a predetermined spark voltage; and
an electromagnet,
wherein the pressure chamber comprises:
a non-compressive fluid contained therein;
a fixed electrode disposed in the non-compressive fluid;
a movable electrode disposed in the non-compressive fluid and separated from the fixed electrode by a predetermined distance; and
a permanent magnet disposed on the movable electrode,
wherein the circuit is configured to apply the spark voltage between the movable electrode and the fixed electrode,
wherein the electromagnet is configured to apply a magnetic force to the permanent magnet to move the movable electrode toward the fixed electrode for generating spark between the movable electrode and the fixed electrode,
wherein the pressure chamber comprises a pressure transferring structure constituting a part of a wall of the pressure chamber and configured to transfer a pressure of the non-compressive fluid generated by the spark to the outside of the pressure chamber.

2. The spark generator according to claim 1, wherein the pressure transferring structure is an elastic membrane.

3. The spark generator according to claim 1, wherein the pressure transferring structure is a piston.

4. The spark generator according to claim 1, wherein the pressure chamber further comprises a selective gas filter configured to release gas generated by decomposition of the non-compressive fluid by the spark to outside of the pressure chamber.

5. The spark generator according to claim 1, wherein the circuit comprises a relay, and
wherein the relay is configured to apply a current to the electromagnet and to apply the spark voltage between the movable electrode and the fixed electrode in a synchronized manner.

6. A formula injector comprising:
a spark generator according to claim 1; and
a formula chamber configured to receive pressure transferred by the pressure transferring structure,
wherein the formula chamber comprises:
a formula contained therein; and
a nozzle configured to inject the formula,
wherein the formula is configured to be injected from the nozzle by the pressure transferred by the pressure transferring structure.

7. The formula injector according to claim 6, wherein the pressure transferring structure is in direct contact with the formula.

8. A method for generating pressure by a spark, comprising the steps of:
applying a predetermined spark voltage between a fixed electrode disposed in a pressure chamber containing a non-compressive fluid and a movable electrode, disposed in the pressure chamber, separated from the fixed electrode at a predetermined distance, and comprising a permanent magnet disposed thereon;
applying a current to an electromagnet disposed in a main device disposed adjacent to the pressure chamber and configured to apply a magnetic force to the permanent magnet to move the movable electrode toward the fixed electrode via the permanent magnet to generate a spark between the movable electrode and the fixed electrode; and
transferring pressure generated in the non-compressive fluid by the spark to the outside of the pressure chamber via a pressure transferring structure.

9. The method according to claim 8, wherein the pressure transferring structure is an elastic membrane, and wherein the pressure is transferred to the outside of the pressure chamber via a deformation of the elastic membrane.

10. The method according to claim 8, wherein the pressure transferring structure is a piston, and wherein the pressure is transferred to the outside of the pressure chamber via a movement of the piston.

11. The method according to claim 8, further comprising releasing gas generated by decomposition of the non-compressive fluid by the spark to the outside of the pressure chamber via a selective gas filter disposed on the pressure chamber.

12. The method according to claim 8, wherein the step of applying the predetermined spark voltage between the fixed electrode and the movable electrode and the step of applying the current to the electromagnet to move the movable electrode toward the fixed electrode via the permanent magnet for generating the spark between the movable electrode and the fixed electrode are carried out in a synchronized manner.

13. A method for injecting a formula, comprising the steps of:
transferring pressure to a formula chamber configured to receive the pressure transferred to the outside the pressure chamber by a method according to any one of claims 8 to 12 via the pressure transferring structure; and
injecting a formula disposed in the formula chamber to the outside of the formula chamber via a nozzle disposed on the formula chamber by the transferred pressure.

14. The method according to claim 13, wherein the pressure transferring structure is in contact with the formula, and
wherein the pressure is directly transferred to the formula.
